(19) United States
(12) Patent Application Publication
(10) Pub. No.: US 2025/0352374 A1
(43) Pub. Date: Nov. 20, 2025

(54) SLEEVE FOR A CATHETER

(71) Applicant: BIOTRONIK AG, Buelach (CH)

(72) Inventors: Karsten Koop, Rostock (DE); Stephan Rothstock, Berlin (DE); André Hein, Schwaan (DE); Paul Goebel, Rostock (DE); Imanol Flores, Rostock (DE); Andreas Hof, Luebeck (DE)

(21) Appl. No.: 19/288,136

(22) Filed: Aug. 1, 2025

Related U.S. Application Data
(63) Continuation of application No. 17/632,002, filed on Feb. 1, 2022.

(30) Foreign Application Priority Data
Aug. 5, 2019 (EP) 19190021.6

(51) Int. Cl. A61F 2/915; A61F 2/82; A61F 2/962

(52) U.S. Cl. CPC A61F 2/915; A61F 2/962; A61F 2002/825; A61F 2002/91525

(57) ABSTRACT

A sleeve for a catheter includes a plurality of webs that extend in a longitudinal direction and are connected to one another by an undulating web in a circumferential direction to form a main web. A through gap is in least one web of the plurality of webs, and the at least one web extends in the longitudinal direction away from the gap on each side of the gap. A bridge web is connected to the at least one web only distally and proximally to the at least one web on each side of the gap. The bridge web is free to flare in the circumferential direction.

### PRIORITY CLAIM AND REFERENCE TO RELATED APPLICATION

The application is a continuation of and claims priority under 35 U.S.C. § 120 and all applicable statutes and treaties from prior pending U.S. application Ser. No. 17/632,002, filed Feb. 1, 2022, which application is a 35 U.S.C. 371 US National Phase and claims priority under 35 U.S.C. § 119, 35 U.S.C. 365 (b) and all applicable statutes and treaties from prior PCT Application PCT/EP2020/071119, which was filed Jul. 27, 2020, which application claimed priority from European Application Serial Number 19190021.6, which was filed Aug. 5, 2019. All applications mentioned in this paragraph are incorporated by reference herein.

### FIELD

Fields of the invention include catheter sleeves for medical implants and medical implants such as stents.

### BACKGROUND

Medical implants, in particular intraluminal endoprostheses, for a wide variety of applications are known from the state of the art in great diversity. Implants within the meaning of the present invention are endovascular prostheses or other endoprostheses, for example stents (stents for vessels (vascular stents, including stents for use in the area of the heart and heart valve stents, such as mitral valve stents, pulmonary valve stents) and bile duct stents), endoprostheses for closing a patent foramen ovale (PFO), stent grafts for treating aneurysms, endoprostheses for closing an atrial septal defect (ASD), and prostheses in the area of hard and soft tissues.

Such an implant usually assumes two states, namely a compressed state having a small diameter and an expanded state having a larger diameter. In the compressed state, the implant can be inserted into the vessel or organ to be treated through narrow vessels by a catheter and positioned at the site to be treated. In the expanded state, the implant remains in the vessel or organ and is secured there after the catheter has been removed from the body of the treated patient. In the case of a transcatheter aortic valve implantation (TAVI, endovascular aortic valve replacement), for example, an artificial aortic valve is introduced into the heart in a tubular support member. The valve is brought into position by catheters. Afterwards, the valve is unfolded and anchored. The endogenous aortic valve is not removed, but displaced by the implant. In the case of a self-expanding implant made of a shape memory alloy, the implant automatically transitions into the expanded state when a transformation temperature is exceeded or a certain amount of stress is exerted. A balloon is required for this purpose in the case of an implant including a balloon-expandable basic support member (stent).

A catheter for releasing a heart valve implant is known from document US 2008/0188928 A1, including a capsule sleeve for advancing the folded heart valve implant through the patient's vasculature, which, on the one hand, is flexible to be guided through the tortuous vessels, and, on the other hand, is suitable for receiving and holding the implant and allowing the implant to be released at the treatment site. The sleeve is composed of an inner polymer layer and an outer polymer layer, between which a support element is or multiple support elements are arranged, which have variable axial stiffness. A tubular support element is formed, for example, of a plurality of rings or ribs, which are arranged next to one another in the longitudinal direction. All ribs are connected by a web that extends in the longitudinal direction.

Documents EP 2 591 751 A1, EP 2 679 198 A1 and US 2010/0249905 A1 show implants that have different discontinuous tubular structures. Document EP 2 679 198 A1 describes a stent for a heart valve implant composed of a wire structure that has multiple portions which are arranged next to one another in the longitudinal direction and which each differ from one another in terms of design and the properties thereof, and which are connected to one another. In contrast, document US 2010/0249905 A1 relates to an implant that has a tubular design and includes a plurality of webs, which are connected by obliquely extending, flexible connectors. The webs and connectors have openings, which are filled with a pharmaceutical drug to be released at the site in the body at which the implant is inserted.

EP 2 591 751 A1 describes an endoluminal prosthesis system for a branched body lumen including a vessel prosthesis (11). The vessel prosthesis (11) can be deployed within a branched vessel lumen and includes a stent (48), which has a generally tubular body portion (33), a flareable proximal end portion (36), and a coupling portion (38) that is arranged between the body portion and the flareable portion. The coupling portion is preferably more crush-resistant than the body portion.

Today, primarily catheters made of plastic materials or composites are used for the implantation of stent-based heart valve implants, which have limited pliability and flexibility. During the implantation and positioning of the heart valve implant, the implant is released from a catheter sleeve (also referred to as a capsule), which held the implant in the compressed state as it was advanced through the patient's vasculature. Such heart valve implants are composed of a support member, which is configured in the manner of a stent and carries the actual valve material. This support member or the stent is designed to be self-expanding, for example, made of a shape memory material such as Nitinol, and is held in the compressed state thereof by the catheter sleeve. As a result of a relative movement of the catheter sleeve with respect to the self-expanding heart valve implant, the compressing force is eliminated, and the self-expanding stent or the support member, and thus the entire heart valve implant, switches from the compressed state to the expanded state. However, the implant also has to be partially retracted into the catheter sleeve to allow the implant to be repositioned.

In particular in the case of self-expanding heart valve implants, strong radial and axial forces arise when the implant is being released and retracted into the catheter sleeve, which are dependent on the stiffness of the implant. These reactive forces can result in permanent deformations of the catheter sleeve, which can cause injuries to the vessels and corresponding complications when the catheter is removed with the catheter sleeve from the body of the treated patient. Greater flexural elasticity is also desirable with implants.

### SUMMARY

A preferred embodiment is sleeve for a catheter. The sleeve includes a plurality of webs that extend in a longitudinal direction and are connected to one another by an undulating web in a circumferential direction to form a main web. A through gap is in least one web of the plurality of webs, and the at least one web extends in the longitudinal direction away from the gap on each side of the gap. A bridge web is connected to the at least one web only distally and proximally to the at least one web on each side of the gap. The bridge web is free to flare in the circumferential direction.

### BRIEF DESCRIPTION OF THE DRAWINGS

FIG. 1 shows a catheter according to the invention prior to the implantation of an implant in a perspective view from the side.

FIG. 2 shows a distal portion of the catheter from FIG. 1 in a perspective view from the side after the implant has been released.

FIG. 3 shows a catheter sleeve according to the invention including an outer shaft in a perspective view from the side;

FIG. 4 shows a cross-section through the catheter sleeve according to FIG. 3 in location C (see FIG. 3).

FIGS. 5-6 each show a distal portion of the catheter sleeve according to FIG. 3 in a view from the side.

FIG. 7 shows the distal portion of a stiffening sleeve of a further exemplary embodiment of a catheter sleeve according to the invention in a view from the side.

FIG. 8 shows a section of the structure according to the invention of the stiffening sleeve of the catheter sleeve according to FIG. 3 in a view from the side.

FIG. 9 shows a section of the structure at the distal end of the stiffening sleeve of the exemplary embodiment shown in FIG. 7 of a catheter sleeve according to the invention in a view from the side.

FIG. 10 shows the joint element of the structures according to the invention shown in the section in FIGS. 8 and 9 in a view from the side.

FIGS. 11-15 show further exemplary embodiments of joint elements of structures according to the invention in a view from the side.

FIGS. 16-17 show an implant, for example in the form of a stent or a stent portion, in which a joint element is integrated into the tubular structure.

### DETAILED DESCRIPTION OF THE PREFERRED EMBODIMENTS

According to the invention, the tubular discontinuous structure for a catheter sleeve or for an implant including a plurality of webs that extend at least partially in the longitudinal direction or circumferential direction includes at least one joint element, the main web of which extending in the longitudinal direction has a severed through-gap (separation). Furthermore, at least one bridge web, and preferably two bridge webs are provided at the joint element, wherein each bridge web is arranged next to the main web in the circumferential direction and connected to the main web in the longitudinal direction in front of and behind the gap.

The main web is completely severed in the region of the gap. The gap is preferably arranged approximately in the center of the joint element. The bridge web acts, or the bridge webs act, as connectors and prevents or prevent the main web from drifting apart under minor tensile forces. The joint element is connected to the webs of the structure, preferably, as viewed in the longitudinal direction, to one web of the structure extending in the longitudinal direction at each of the two ends, particularly preferably in the region in which the bridge web is, or the bridge webs are, connected to the main web. In a particularly preferred exemplary embodiment, this connecting web forms the continuation of the main web in the longitudinal direction.

Within the scope of the present invention, webs extending in the longitudinal direction shall be understood to mean webs that run both exactly parallel and at a small angle with respect to the longitudinal direction of the structure. When the structure located on the circumference of the tube carry out a rolling motion in a plane, the angle between the web and the longitudinal direction is no more than 0 to 45°. The longitudinal direction of a structure, such as with a substantially cylindrical implant or a catheter sleeve, corresponds to the cylinder axis of the cylindrical implant structure or the catheter axis of a catheter sleeve.

The joint element according to the invention is a structure that, in terms of the properties thereof, is comparable to a solid-state hinge and able to transmit pressure forces similarly to human joints, but also allows sliding in the region of the gap that is arranged in the main web and twisting of the ends of the main web, which are located opposite one another in the gap, with respect to one another. The joint element therefore has high flexural elasticity. The joint element is further able to transmit pressure forces that run in the longitudinal direction or in the radial direction. This takes place, on the one hand, via the bridge webs. On the other hand, when a predefined axial pressure is exceeded, the ends of the main web which form the gap are compressed so as to bear on one another. The transmission of pressure then takes place not only via the bridge web or webs, but also via the main web. The joint element according to the invention, of which a plurality are preferably present in the structure, imparts the desired flexural elasticity to the structure, wherein it is also possible to transmit high radial and axial pressure forces. In the case of stent implants, the joint structure can also be used at the web intersecting points in the circumferential direction. The stent is thereby given high flexural elasticity, which facilitates the adaptation to anatomic structures, such as calcifications. The radial force can thus be transmitted by way of the closing joints, while the stent has high flexibility (low crush resistance) in the circumferential direction, which is necessary to be particularly adaptable.

In a preferred exemplary embodiment, the bridge web is semi-circular, U-shaped, V-shaped or meander-shaped. The different shapes of the bridge webs allow an adaptation to the different flexural elasticity required by the structure, as a function of the use of the structure. In a further embodiment, the bridge webs can include additional spring elements or can include changes in the cross-section. Greater flexural elasticity can be achieved by the bridge web including at least one notched portion having a notch that extends transversely to the longitudinal direction. Such a notched portion can be designed as a U-shaped, V-shaped or W-shaped portion. As an alternative, the bridge web may only include a region having a reduced width or thickness in the portion.

It is advantageous when the gap has a width of at least 20 μm, preferably of at least 40 μm to 500 μm, depending on the application. In the process, the gap width is measured in the direction of the longitudinal axis (that is, in the longitudinal direction) of the tubular structure. In this way, the desired increased flexural elasticity is ensured.

It is likewise advantageous for the flexural elasticity of the joint element when the bridge web has a width of 20% to 40% of the main web width. In embodiments that include more than one bridge web, the width of the bridge webs is selected so as not to exceed, in sum, the width of the main web. Accordingly, in an embodiment that includes two bridge webs, the bridge webs preferably each have a width of 20% to 50% of the main web width. In an embodiment that includes only one bridge web, the bridge web has a width of 20% to 100% of the main web. The width is measured in each case perpendicularly to the center line of the respective web.

Flexural elasticity in any given radial direction is ensured when the structure includes a plurality of joint elements, which are arranged next to one another in the circumferential direction. The plurality of joint elements is particularly preferably provided along the entire circumference of the structure, so that a funnel-shaped radial expansion of the structure is achieved, for example when a self-expanding implant is being released. With respect to a catheter sleeve, it is in particular advantageous when a portion including a plurality of joint elements that are arranged next to one another in the circumferential direction forms a distal portion of the catheter sleeve since increased flexural elasticity is required in this region, in particular in the distal portion of the catheter sleeve, for releasing and retracting, for example, a heart valve implant from/into the catheter. In the longitudinal direction, particularly preferably at least two such portions, including a plurality of joint elements, are provided consecutively in the longitudinal direction. The joint elements can be designed in such a way that the flexibility thereof decreases in the proximal direction, for example due to reinforcement of the bridge webs. In a preferred exemplary embodiment of the structure according to the invention, a portion is provided, in the longitudinal direction, adjoining the portion including the plurality of joint elements that are arranged next to one another, in which one or more undulated webs are arranged, which particularly preferably extend around the entire circumference. These can absorb the forces that are passed on by the joint elements. In this way, problematic deformation of the catheter sleeve is avoided when the catheter is guided out of the body.

In an exemplary embodiment of the present invention, it is advantageous when the structure according to the invention is made of a shape memory alloy, in particular Nitinol, or includes the same. A structure according to the invention made of polymer, a cobalt-chromium alloy (CoCr) or steel can likewise be expedient in certain embodiments.

The above object is also achieved by an implant, in particular a stent, that includes the above-described tubular, discontinuous structure according to the invention at least in one portion. In this portion, the implant has the described flexural elasticity, wherein it is also possible to transmit high pressure forces in the radial and axial directions. According to the invention, the implant can also be made entirely of the described tubular, discontinuous structure including a joint element, or a plurality of joint elements, which are arranged behind one another or next to one another, both in the circumferential direction and in the longitudinal direction. Especially in the case of heart valve stents, high adaptability in the circumferential direction can be advantageous to adapt to anatomical structures, such as calcifications.

The above object is further achieved by a catheter sleeve, which is suitable, in particular, for the introduction of a stent-based heart valve implant. The catheter sleeve according to the invention includes a stiffening sleeve and a first polymer layer, which is arranged within the stiffening sleeve in the radial direction. Furthermore, a second polymer layer is provided, which is arranged outside the stiffening sleeve in the radial direction, wherein the above structure according to the invention forms a distal portion of the stiffening sleeve, which is also referred to as a crown. In particular in the region of the crown, that is, in the region of the structure according to the invention, the small stiffening tube is made of a shape memory alloy, preferably Nitinol. A stiffening sleeve shall accordingly be understood to mean a mechanically stable structure that, as part of the catheter sleeve, covers the implant, such as the stent-based heart valve implant, when the catheter is inserted, and, in the case of a self-expanding implant, maintains the compressed shape of the implant.

Due to the flexural elasticity, the catheter sleeve according to the invention, also referred to as an implant capsule, allows the catheter sleeve to be flared at the distal end so as to release the implant arranged therein. In addition, it is also possible to transmit axial pressure forces during resheathing, so that the catheter sleeve, after resheathing, returns completely to the initial shape thereof. In particular, the structure can be flared similarly to a trumpet or a funnel when the crown, along the entire circumference thereof, includes a plurality of the above-described joint elements, which are arranged next to one another. The bridge elements cause the joint element to be guided and determine the pliability thereof. However, they are also able to transmit minor tensile forces so as to prevent the joint element from tearing apart.

In a preferred exemplary embodiment, the catheter sleeve according to the invention includes at least two portions that are arranged behind one another in the longitudinal direction, wherein a plurality of joint elements are arranged next to one another in the circumferential direction in each portion, distributed across the entire circumference. The catheter sleeve according to the invention allows an implant to be released and retracted without difficulty, and thereafter returns to the initial shape thereof without difficulty, so that deformations, and thus undesirable interactions with the vessel when the catheter sleeve is being guided out, are avoided.

The above object is achieved analogously by a catheter including the above-described catheter sleeve, wherein the catheter sleeve is used and designed to receive a folded implant, in particular a stent-based heart valve implant, and is connected to the outer shaft of the catheter. The implant is preferably fixed on the inner shaft of the catheter by a so-called prosthesis connector. As with conventional catheters, the outer shaft is guided and movable on the inner shaft.

Further objectives, features, advantages, and application options of the invention will also be apparent from the following description of exemplary embodiments of the invention based on the figures. All features that are described and/or illustrated, either alone or in any arbitrary combination, form the subject matter of the present invention, also independently of their combination in the individual claims or their dependency reference.

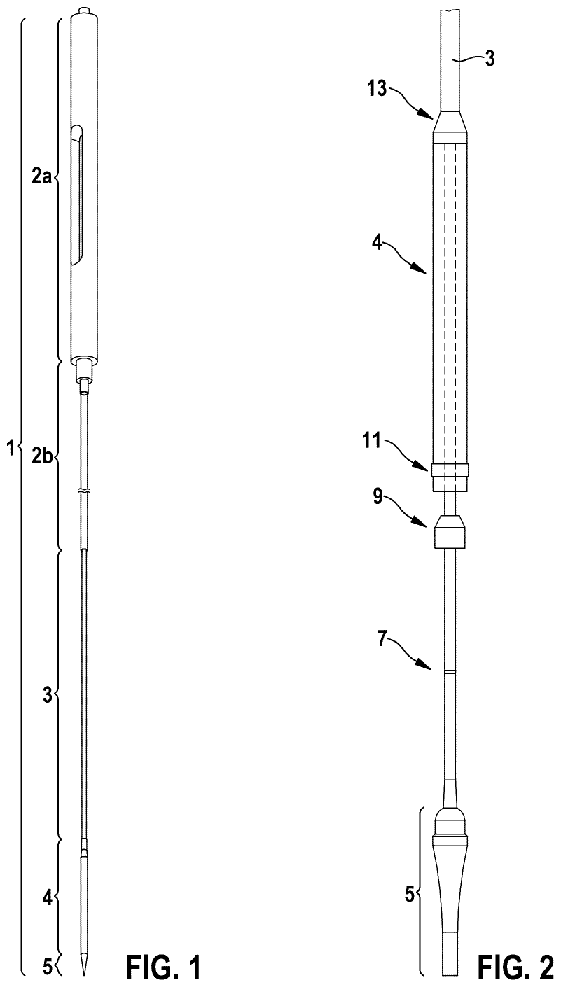

FIG. 1 shows a catheter 1 according to the invention, including a handle 2a arranged at the proximal end of the catheter, a stabilization portion 2b, an outer shaft 3, and a catheter sleeve 4 arranged on the outer shaft 3, such as is used, for example, for implanting a self-expanding stent-based heart valve implant. A dull catheter tip 5 is provided at the outermost distal end. The stabilization portion 2 shields the retractable outer shaft 3 with respect to the insertion sheath (introducer) and the vessel wall, so that the outer shaft 3 can be freely retracted. The handle 2a is used to load, release and retract an implant that is arranged in the catheter sleeve 4, for example of a stent-based heart valve implant. The catheter tip 5 forms the distal end of an inner shaft 7 arranged within the outer shaft 3 (see FIG. 2), wherein the catheter tip 5 is preferably made of PEBAX and visible when irradiated with X-rays.

FIG. 2 represents the distal end of the system illustrated in FIG. 1 after the implant has been released. This figure also shows that a prosthesis connector 9, by which the implant is fixed axially to the inner shaft 7, is arranged on the inner shaft 7. At the distal end, the catheter sleeve 4 preferably includes a ring 11 that is visible when irradiated with X-rays to facilitate monitoring. The catheter sleeve 4 is connected to the outer shaft 3 by a proximal connector 13. The catheter sleeve 4 and the outer shaft 3, however, can also be designed in one piece in other embodiments.

As was already described above, in the state shown in FIG. 1, the implant is initially arranged in the catheter sleeve 4 (also referred to as a capsule) in the compressed state and is held in this state by the catheter sleeve 4. The catheter sleeve 4 is connected to the handle 2a by the outer shaft 3. In this state, the compressed implant fixed in the catheter sleeve 4 is advanced through the vessels of the patient to the treatment site.

The catheter sleeve 4 is pulled toward the proximal end to release the implant. The retraction is triggered by the handle 2a and transferred onto the catheter sleeve 4 by the outer shaft 3. Initially, only a short distal portion of the implant is released, and the fit is checked. If the positioning is unfavorable, the catheter sleeve is pushed toward the distal end again by the handle 2a, whereby the implant is covered by the catheter sleeve 4 again and has transitioned completely into the compressed state. The catheter 1 is now repositioned, and the release of the implant arranged in the catheter sleeve 4 starts again. So as to avoid deformations of the catheter sleeve 4 during the release, and possibly during the retraction, of the catheter sleeve 4, the catheter sleeve has to be particularly flexible and additionally be able to transmit axial and radial pressure forces well.

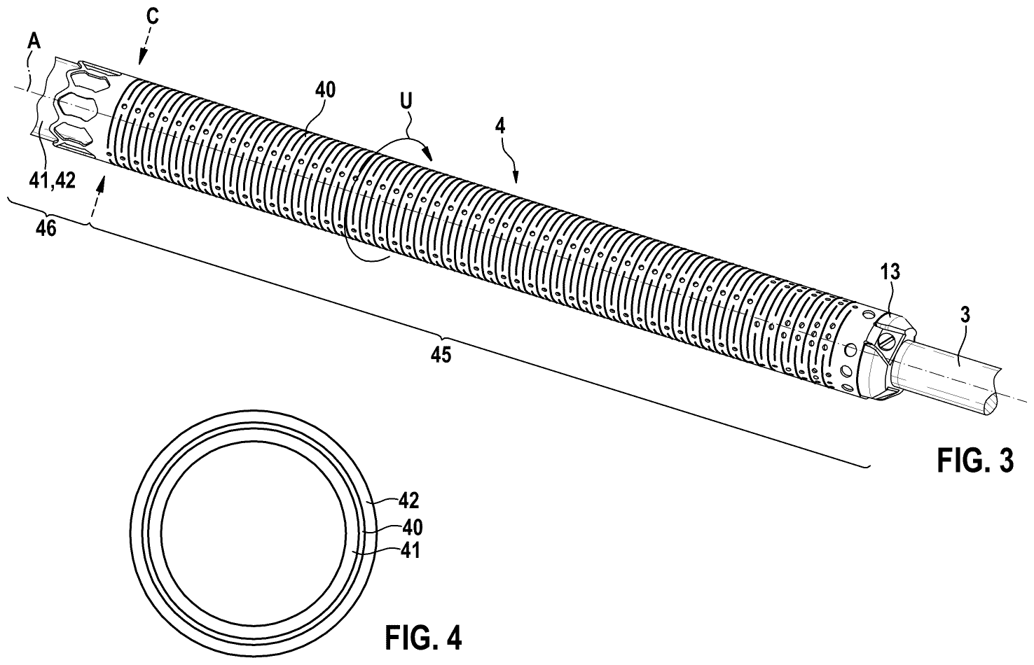

The catheter sleeve 4 is composed of a stiffening sleeve 40, which is embedded between an inner first polymer layer 41 and an outer second polymer layer 42 surrounding the stiffening sleeve 40. The polymer layers 41, 42 surround the stiffening sleeve 40 and, at the distal end of the stiffening sleeve, protrude beyond the distal end of the stiffening sleeve 40. The stiffening sleeve 40 is preferably made of a metallic material (alternatively, stiff polymer material) and includes a proximal portion 45 as well as a distal portion 46, wherein the distal portion is also referred to as a crown. The proximal portion 45 is particularly preferably a stainless steel sleeve, which is partially slotted. At the outermost proximal end of the proximal portion 45, the stiffening sleeve 40 is connected to the outer shaft 3 by the proximal connector 13. The center line of the stiffening sleeve 40 forms the longitudinal direction A (see FIG. 3) of the stiffening sleeve 40 or of the catheter sleeve 4.

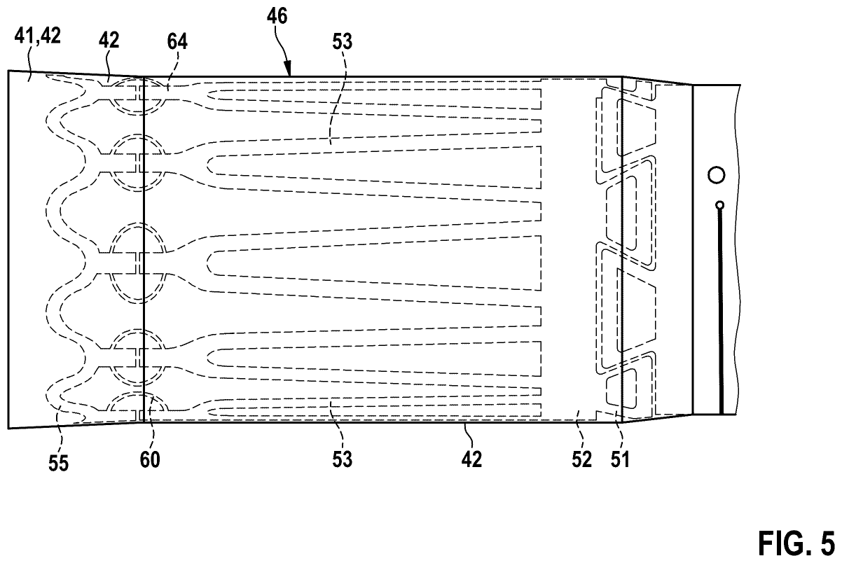

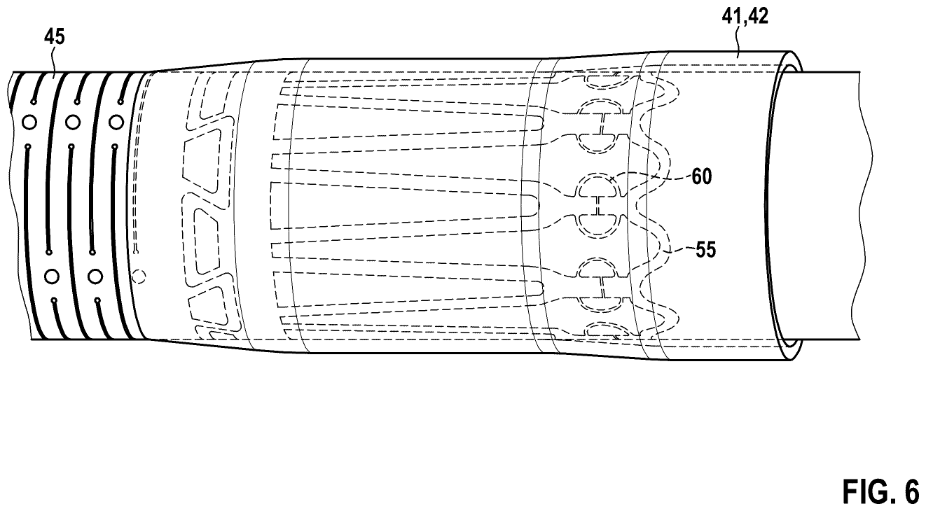

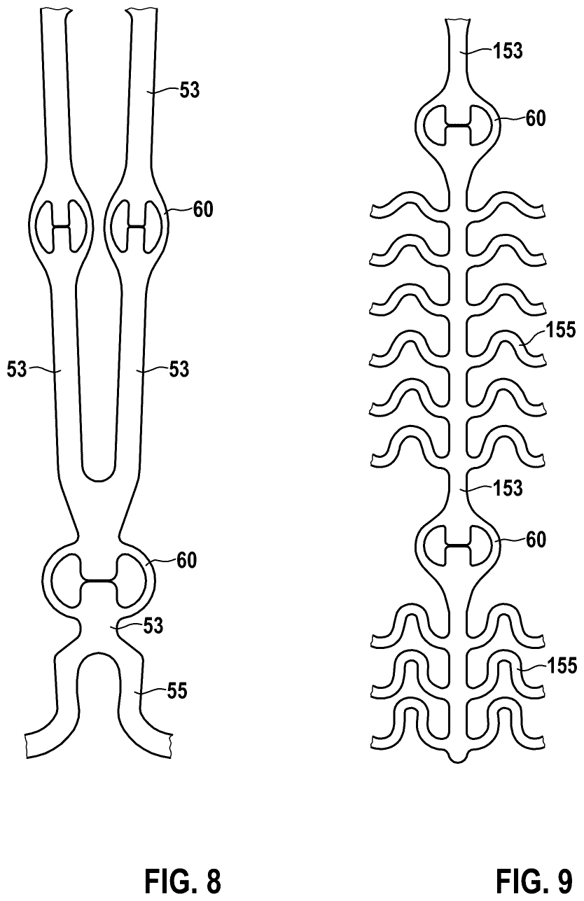

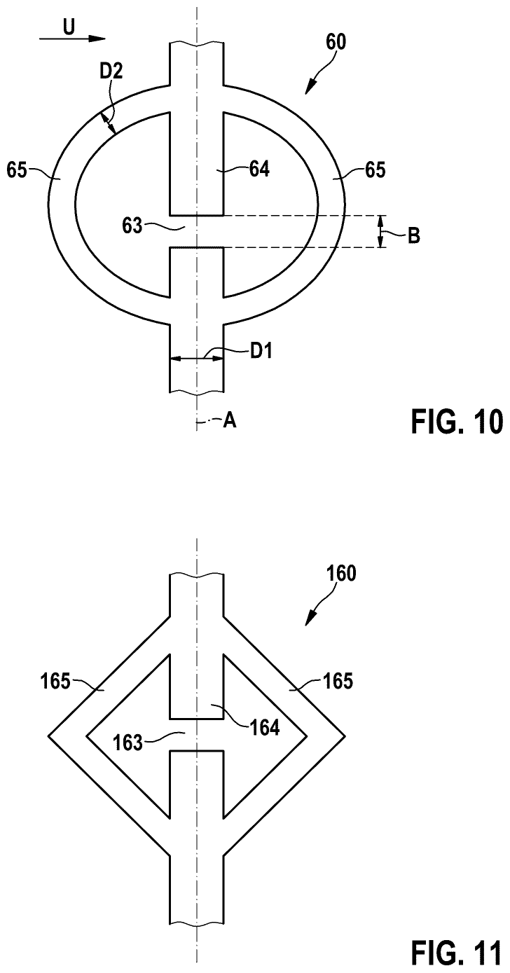

The distal portion 46 of the stiffening sleeve 40 is shown in greater detail in FIGS. 5 and 6 as well as in a section in FIG. 8 in an enlarged illustration. The distal portion 46 made of Nitinol includes dovetail-shaped webs 51 at the proximal end thereof, which are engaged with corresponding dovetail-shaped notches of the proximal portion 45. In the adjoining portion in the distal direction, the structure forms a ring 52, which in the distal direction is connected to webs 53 that extend substantially in the longitudinal direction. At the distal end, these webs 53 extending in the longitudinal direction are connected to one another by an undulated web 55 extending around the entire circumference of the stiffening sleeve 40 and extending in the circumferential direction U. The webs 53 that extend in the longitudinal direction are wider distally and extend in the proximal direction in the shape of a two-tine fork. In the region of the larger width thereof, each web 53 extending in the longitudinal direction includes a joint element 60, which is shown in detail again in FIGS. 8 and 10. FIG. 8 shows an alternative exemplary embodiment in which the narrower fork webs 53, which extend in the longitudinal direction A, also each include a joint element 60.

The joint element 60 shown in detail in FIG. 10 includes a main web 64 extending in the longitudinal direction A (corresponds to the longitudinal direction of the tubular structure of the stiffening sleeve 40 or of the catheter sleeve 4). In the structure according to the invention, the main web 64 forms a continuation of the respective web which extends in the longitudinal direction and is denoted by reference numeral 53. The main web 64 is completely severed centrally in the region of the joint element 50, whereby a gap 63 is created. A respective bridge web 65 is arranged on each side in the circumferential direction U next to the gap 63 or the main web 64, which in each case approximately forms a semi-circular shape and is connected to the main web 64 in the distal and proximal directions in front of and behind the gap. The joint element 60 allows axial pressure forces, that is pressure forces that extend in the longitudinal direction A, as well as radial pressure forces to be transmitted, so that the distal portion 46 enables a flaring of the structure in the shape of a funnel. The joint element 60 is less stiff in the stretching direction, however the bridge web(s) 65 prevent(s) the structure of the distal portion 46 from drifting apart under minor tensile forces. Depending on the width B of the gap 63 of 40 μm to 500 μm, a spring function having a stop (maximum force) can additionally be implemented. In this way, the catheter sleeve 4 according to the invention has particularly good flexible properties during the release or retraction of an implant and completely returns elastically to the initial shape thereof, so that deformation can be avoided.

In the exemplary embodiment shown here, the main web 64 has a width D1 (measured in the circumferential direction U) of 50 μm to 500 μm, and each bridge web 65 has a width D2 of 20% to 50% of the web width of the main web (measured perpendicularly to the center line of the bridge web 65).

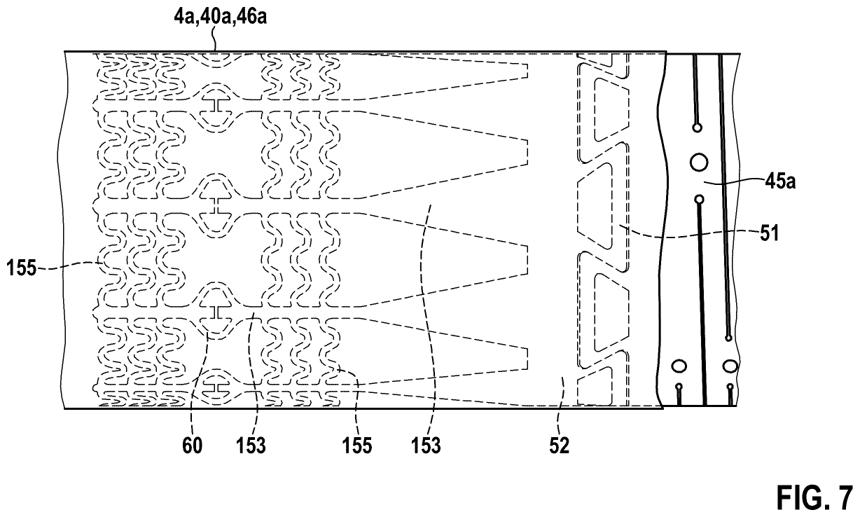

FIGS. 7 and 9 relate to a second exemplary embodiment of a catheter sleeve 4a according to the invention including a slightly modified stiffening sleeve 50a. Similarly to the distal portion 46 of the preceding exemplary embodiment, the distal portion 46a includes dovetail-shaped webs 51 at the proximal end for the connection to the proximal portion 45a of the stiffening sleeve 40a. A ring 52 adjoins in the distal direction, which by way of appropriate notches gradually transitions into webs 153 that extend in the longitudinal direction A. The webs 153 extending in the longitudinal direction are connected to one another by way of a plurality of undulated webs 155 that extend in the circumferential direction U. Joint elements 60, which are arranged at each web 153 extending in the longitudinal direction and are located next to one another in the circumferential direction U, are provided at the distal end of the distal portion 46a between two portions including undulated webs 155. The joint element 60 is shown in FIG. 10 and was already described above.

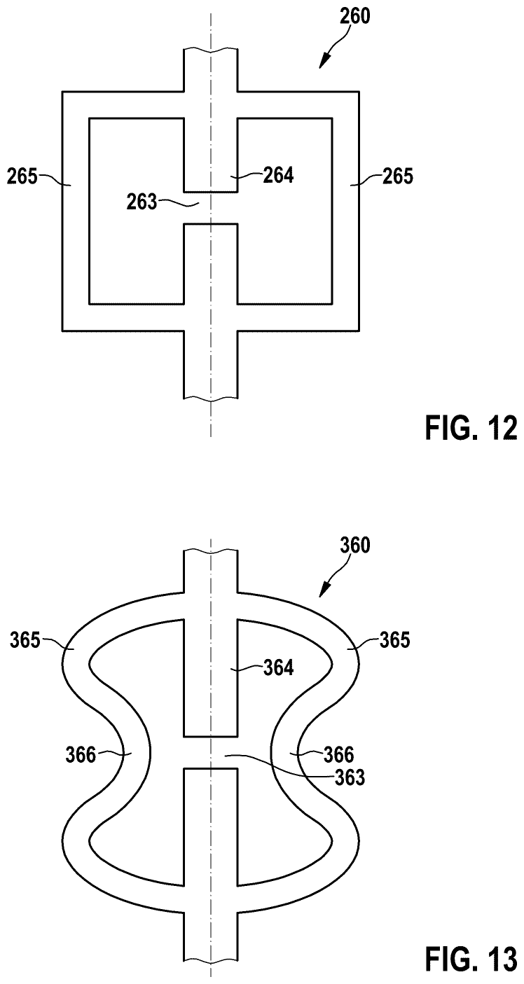

FIGS. 11 to 15 show further exemplary embodiments of joint elements. In contrast to the joint element 60 of FIG. 10, the joint element 160 shown in FIG. 11 includes V-shaped bridge elements 155. In the exemplary embodiment of a joint element 250 shown in FIG. 11, the bridge web 255 has a U-shaped configuration.

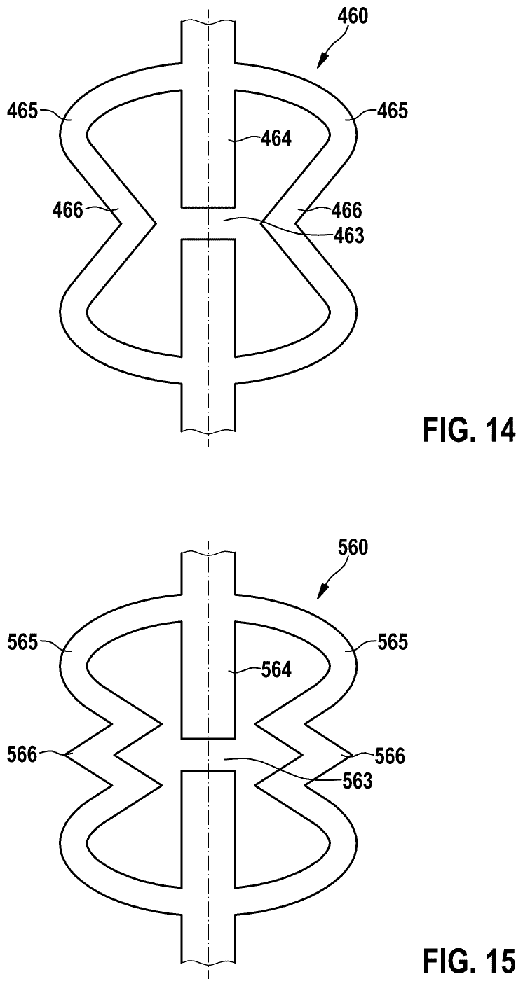

The exemplary embodiments of joint elements 350, 450, 550 provided in FIGS. 13 to 15 resemble the joint element 50 shown in FIG. 10, but include a portion approximately at the height of the gap 355, 455, 555 in the region of the respective bridge web 365, 465, 565, which has a notch for increasing the flexibility. This notched portion may (not shown) only encompass a decrease in the width or thickness of the respective bridge web 365, 465, 565 or, as shown in the figures, a change in direction of the bend. This results in a U-shaped notched portion 366 (or a wave shape, see FIG. 13), or a V-shaped notched portion 466 (FIG. 14). This portion is rather rounded in FIG. 13, and it is rather pointed in FIG. 14. In FIG. 15, the notched portion 566 has a W-shaped design, which increases the spring property of the bridge web 565.

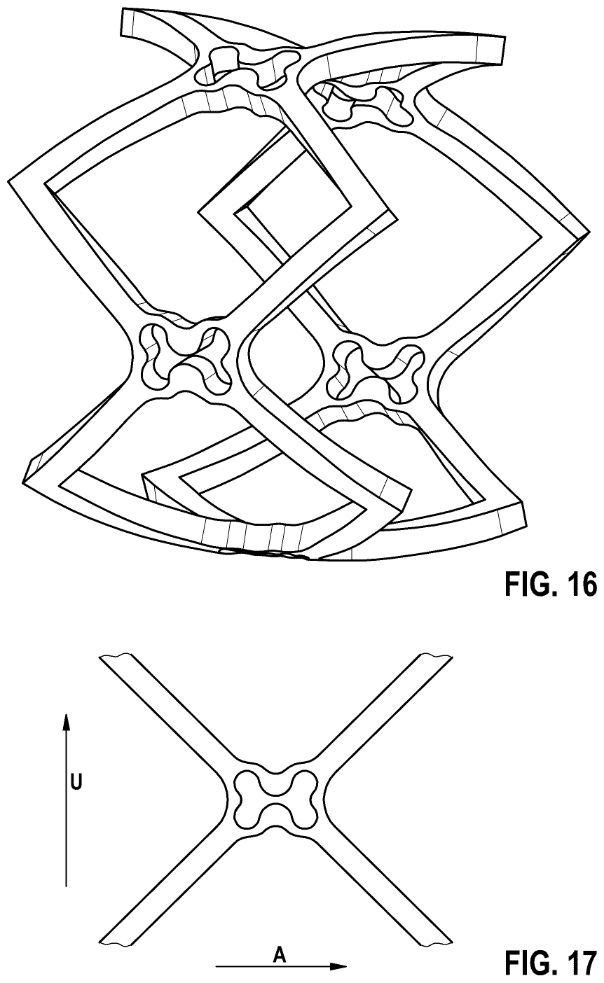

For implants such as stents, embodiments are also conceivable which include joints aligned in the circumferential direction U in such a way that the main web (or node) is oriented in the circumferential direction U, and a joint gap is interrupted thereby. Such an embodiment is shown as a stent or a section of a stent in FIG. 16. The exact configuration of this example of the joint can be derived from FIG. 17. It becomes evident that the sectional shape of the joint resembles a shortened bone or two hearts superimposed at the apexes. The joints can also be used as a replacement for strut intersecting points to increase the flexural elasticity in the longitudinal and circumferential directions.

The above-described structure according to the invention, which can be used in a catheter sleeve or in an implant, allows high radial and axial pressure forces to be transmitted, while ensuring high flexural elasticity at the same time.

While specific embodiments of the present invention have been shown and described, it should be understood that other modifications, substitutions and alternatives are apparent to one of ordinary skill in the art. Such modifications, substitutions and alternatives can be made without departing from the spirit and scope of the invention, which should be determined from the appended claims.

Various features of the invention are set forth in the appended claims.

### LIST OF REFERENCE NUMERALS

1 catheter
2a handle
2b stabilization portion
3 outer shaft
4, 4a catheter sleeve
7 inner shaft
9 prosthesis connector
11 radio-opaque ring
13 proximal connector
40, 40a stiffening sleeve
41 first polymer layer
42 second polymer layer
45, 45a proximal portion
46, 46a distal portion
51 dovetail-shaped web
52 ring
53, 153 web extending in the longitudinal direction
55, 155 undulated web
60, 160, 260, 360, 460, 560 joint element
63, 163, 263, 363, 463, 563 gap
64, 164, 264, 364, 464, 564 main web
65, 165, 265, 365, 465, 565 bridge web
366, 466, 566 notched portion
(U-shaped, V-shaped or W-shaped)
U circumferential direction
A longitudinal direction of the stiffening sleeve 40 or of the catheter sleeve or of an implant
B width of the gap
D1 width of the main web 64
D2 width of the bridge web 65

1. A sleeve for a catheter comprising:
a plurality of webs that extend in a longitudinal direction and are connected to one another by an undulating web in a circumferential direction to form a main web;
a through gap in least one web of the plurality of webs, the at least one web extending in the longitudinal direction away from the gap on each side of the gap; and
a bridge web connected to the at least one web only distally and proximally to the at least one web on each side of the gap.

2. The sleeve according to claim 1, wherein the bridge web is free to flare in the circumferential direction.

3. The sleeve according to claim 1, wherein the bridge web comprises two bridges on opposite circumferential sides of the gap, each of which is connected to the at least one web only distally and proximally to the at least one web on each side of the gap.

4. The sleeve according to claim 1, wherein the bridge web comprises a semi-circular, U-shaped, V-shaped or meander-shape.

5. The sleeve according to claim 1, wherein the bridge web comprises at least one notched portion.

6. The sleeve according to claim 1, wherein the gap has a width (B) of at least 20 μm.

7. The sleeve according to claim 6, wherein the width (B) is in the range of at least 40 μm to 500 μm.

8. The sleeve according to claim 1, wherein a width (D2) of the bridge web is at least 20% of the web width of the main web.

9. The sleeve according to claim 1, wherein each of the plurality of webs comprises a through gap and a bridge web separately connected to each of the plurality of webs only distally and proximally to each of the plurality of webs on each side of the gap, wherein the bridge web of each of the plurality of webs is free to flare in the circumferential direction.

10. The sleeve according to claim 9, wherein the through gap and bridge web of each of the plurality of webs are arranged next to one another in the circumferential direction.

11. The sleeve according to claim 1, wherein the plurality of webs is formed of a shape memory alloy.

12. The sleeve according to claim 11, wherein the shape memory alloy is Nitinol.

13. A catheter sleeve comprising a sleeve of claim 1 and first and second polymer layers inside and outside of the stiffening sleeve.

14. A catheter comprising a catheter sleeve of claim 12, wherein the catheter sleeve is connected to an outer shaft of the catheter and is configured to receive an implant.

15. A catheter sleeve comprising a sleeve of claim 1 to stiffen one or more polymer layers of the catheter sleeve.
